(19) United States
(12) Patent Application Publication
(10) Pub. No.: US 2017/0233826 A1
(43) Pub. Date: Aug. 17, 2017

(54) MRNA AND/OR PROTEIN OF ERCC1 ISOFORM 3 FOR USE IN DIAGNOSING A RESISTANCE AGAINST A THERAPEUTIC AGENT AND METHOD FOR DIAGNOSING A RESISTANCE AGAINST A THERAPEUTIC AGENT USING SAID MRNA AND/OR PROTEIN

(71) Applicant: Adnagen GmbH, Langenhagen (DE)

(72) Inventors: Siegfried HAUCH, Isernhagen-Süd (DE); Yvonne BOKELOH, Nienstädt (DE); Jenny WAGNER, Wedemark (DE)

(21) Appl. No.: 15/511,837

(22) PCT Filed: Sep. 16, 2015
(86) PCT No.: PCT/EP2015/071170

§ 371 Date: Mar. 16, 2017

(30) Foreign Application Priority Data
Sep. 18, 2014 (EP) 14185247.5

(51) Int. Cl. C12Q 1/68

(52) U.S. Cl. CPC C12Q 1/6886; C12Q 1/6804; C12Q 2600/158; C12Q 2600/106; C12Q 2600/156

(57) ABSTRACT

A method for diagnosing a resistance against a therapeutic agent in a patient, which includes measuring an amount of ERCC1 isoform 3 protein and/or an amount of ERCC1 isoform 3 mRNA, which present in a liquid sample or a tissue sample obtained from the patient, and comparing the measured amount to a predetermined value, wherein an amount above the predetermined value indicates resistance against said therapeutic agent.

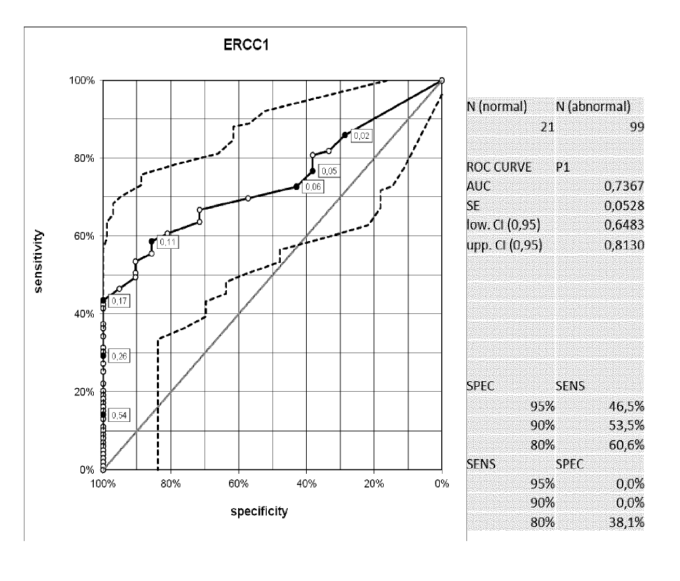

The detection of circulating tumor cells (CTC) is widely accepted as an independent prognostic tool in solid cancers as well in primary disease and meta-static disease. Therapy failure during treatment of said diseases was indicated by CTC not disappearing in the course of a given therapy. However, it turned out that such prognostic information alone is of limited usefulness since it does not help in predicting therapeutic success of certain therapeutic agents before a therapy is even started.

Thus, there is a need for further characterization of CTC in order to identify markers which are present in CTC and may give useful information about the possible success of a therapy with certain therapeutic agents, as well as information on putative therapeutic targets, where a future therapy can individually be adjusted to.

The AdnaTest®, which combines immunomagnetic capturing followed by a molecular characterization of such captured cells by means of mRNA profiling, is a useful tool to address such questions. For example, in ovarian cancer, an early identification of a successful therapy is of paramount importance, especially in ovarian cancers where prognosis is already bad. Such method is disclosed e.g. in EP 1 409 727 A2, which is herewith incorporated by reference.

Platinum-based therapeutic agents or regimens are the first and most promising choice in the treatment of some forms of cancer, e.g. ovarian cancer after surgery. However, about 20% of the patients already have developed, or are about to develop, resistance against platinum-based therapeutics. Thus, said patients no longer benefit from these therapeutics or these regimen.

ERCC1 is a DNA repair gene which was regarded for a long time as a tissue biomarker to provide predictive information about a resistance to platinum-based therapeutic agents. However, it has recently been found that immunohistochemical detection of ERCC1 protein in tissue lacks specificity and was unsuitable for correct prediction of resistance to platinum-based therapeutic agents. It was found that ERCC1 isoforms 2, 3 and 4 are non-functional in nucleotide excision repair capacity. Consequently, it was assumed that they are unsuitable for use as a biomarker. Among the four different isoforms of ERCC1, only ERCC1 isoform 1 was found to be suitable to be used as a biomarker (see Friboulet L. et al., Cell Cycle, vol. 12, p. 3298-3306). However, ERCC1 isoforms 1 and 4 are highly similar which means that it is not possible to date to specifically detect isoform 1 expression without codetection of isoform 4 expression. Importantly, the expression of ERCC1 isoforms 1 and 4 is time and tissue-specific which means a detection of isoform 1 is afflicted with a large inter-tissue variation and is error-prone because of interference with isoform 4 codetection.

Starting herefrom, it was the object of the present invention to provide a biomarker which provides a reproducible and tissue-independent prediction of resistance against a therapeutic agent which is less prone to detection errors.

The problem is solved by ERCC1 isoform 3 protein for use according to claim 1, ERCC1 isoform 3 mRNA for use according to claim 2, the method for diagnosing a resistance against a therapeutic agent according to claim 3 and the use of ERCC1 isoform 3 protein and/or mRNA as biomarker according to claim 4. The dependent claims refer to preferred embodiments thereof.

According to the invention, ERCC1 isoform 3 protein and/or mRNA is suggested for use in diagnosing a resistance against a therapeutic agent. The isoform 3 of ERCC1 is also known as “ERCC1 201” and the protein has the identifier P07992-3 at the “Swiss-Prot” protein database (see www.uniprot.org). With ERCC1 isoform 3 mRNA, any mRNA is understood which codes for the ERCC1 isoform 3 protein having the identifier P07992-3 at the “Swiss-Prot” protein database.

It was surprisingly found that the level of ERCC1 isoform 3 protein production in a cell, as well as the level of ERCC1 isoform 3 mRNA expression in a cell, especially in circulating tumor cells, correlates with resistance to therapeutic agents like platinum-based therapeutic agents. The finding is surprising because the isoform 3 of ERCC1 is known not to be involved in the development of resistance to therapeutic agents. The advantage of ERCC1 isoform 3 protein and/or mRNA is that it is structurally considerably different to the other isoforms of ERCC1 and thus can be determined without the problem of codetection of isoforms 1, 2 and/or 4. Consequently, ERCC1 isoform 3 is a more reliable marker for detecting resistances compared to e.g. ERCC1 isoform 1.

Additionally, a method for diagnosing a resistance against a therapeutic agent is provided. The method comprises or consists of the steps of
a) measuring an amount of ERCC1 isoform 3 protein and/or an amount of ERCC1 isoform 3 mRNA, which present in a liquid sample or a tissue sample, preferably a liquid sample from a human body or a tissue sample from a human body, and
b) comparing the measured amount to a predetermined value, wherein a measured amount above the predetermined value indicates resistance against said therapeutic agent.

The therapeutic agent may be a cancer therapeutic agent, preferably selected from the group comprising or consisting of platinum-based cancer therapeutic agents, radiation-based cancer therapeutic agents and DNA-destructive cancer therapeutic agents.

Additionally, the use of ERCC1 isoform 3 protein and/or ERCC1 isoform 3 mRNA as biomarker for resistance to a therapeutic agent is suggested. Preferably, the biomarker is a biomarker used for detecting a resistance to a cancer therapeutic agent, more preferably a platinum-based cancer therapeutic agent, a radiation-based cancer therapeutic agent and/or a DNA-destructive cancer therapeutic agent.

Regarding the above recited protein, mRNA, method and/or use, the following embodiments are particularly preferred.

Diagnosing may be done on a liquid sample from a human body and/or a tissue sample from a human body, preferably an isolated liquid sample from a human body and/or a tissue sample from a human body (in-vitro-method). The sample may comprise or consist of a body fluid (e.g. peripheral blood, sputum, ascites, lymph, urine and/or bone marrow). The sample may also comprise or consist of a biopsy material (e.g. a biopsy material from a primary tumor).

The cancer may be selected from the group comprising or consisting of a solid cancer, preferably ovarian cancer, breast cancer, lung cancer, prostate cancer, pancreatic cancer, bladder cancer, gastric cancer, colon cancer, testicular cancer and neck cancer. Particularly preferred is ovarian cancer. Most preferably diagnosing regards ovarian cancer after surgery.

Diagnosing the resistance may comprise a step of isolating circulating tumor cells, preferably by a multi-antibody capturing method, and a step of determining the level of expression of ERCC1 isoform 3 protein and/or ERCC1 isoform 3 mRNA in the isolated circulating tumor cells. In this regard, the isolation of the circulating tumor cells may be performed on a liquid sample from a human body and/or a tissue sample from a human body. Preferably, the isolation of the circulating tumor cells is performed on an isolated liquid sample from a human body and/or a tissue sample from a human body (in-vitro-method).

For example, for isolating circulating tumor cells, the AdnaTest® OvarianCancerSelect/Detect may be used.

The step of isolating circulating tumor cells may comprise or consist of the steps of
a) mixing a liquid sample, preferably a liquid sample from a human body, and/or a tissue sample from a human body, containing circulating tumor cells, with a predetermined combination of at least two antibodies and/or antibody derivatives, wherein the antibodies and/or antibody derivatives have a binding affinity to circulating tumor cells; and
b) isolating complexes between i) the antibodies and/or antibody derivatives and ii) the circulating tumor cells, from the sample.

The antibodies and/or antibody derivatives are preferably coupled to at least one magnetic or paramagnetic particle. In this case, magnetic force may be used for isolation of said complexes. Additionally or alternatively, the antibodies and/or antibody derivatives may be coupled to at least one fluorescent molecule. In this case, fluorescence activated cell sorting can be used for isolation of said complexes.

At least one of the two antibodies and/or antibody derivatives may be selected from the group comprising or consisting of GP1.4, MOC-31, Ber-EP-4, HMPV.2, HMEIV.2, 8B6, E29, 10E9 (anti-HER2), 2D3 (anti-EpCAM) anti-cMET, anti-EGFR, anti-cKIT, anti-IGFR and 131-11741.

Diagnosing the resistance may comprise the step of
a) lysing isolated circulating tumor cells,
b) measuring an amount of ERCC1 isoform 3 protein and/or ERCC1 isoform 3 mRNA, present in the lysed isolated circulating tumor cells, and
c) measuring an amount of at least one mRNA which is present in the lysed isolated circulating tumor cells and is different to ERCC1 isoform 3 mRNA, wherein the at least one mRNA is selected from the group comprising or consisting of mRNAs which are markers for cells of epithelial, mesenchymal or stem cell phenotype,
   wherein step b) and step c) may be changed in their sequence.

Preferably, measuring in step b) and/or step c) is done by RT-PCR, LCR, NASBA, a hybridisation method and/or a method containing the steps of PCR, digesting of the PCR product with restriction enzymes to fragments and analysis of said obtained fragments.

The at least one mRNA, which is different to ERCC1 isoform 3 mRNA, may be selected from the group comprising or consisting of mRNAs which are markers for cells of epithelial, mesenchymal or stem cell phenotype, preferably the group comprising or consisting of mRNAs recited in Table 1.

TABLE 1

| epithelial markers | mesenchymal markers | stem cell markers |
|---|---|---|
| CD166 | 5′-Nucleotidase/CD73 | BMI1 |
| Adenosine 5′-Triphosphatase | ALCAM/CD166 | ALDH1 |
| CD138 (Syndecan-1) | Aminopeptidase N/CD13 | CD44 |
| beta-Crystallin | BMPR-IA/ALK-3 | CD24 |
| beta-Defensin 2 | BMPR-IB/ALK-6 | KRT19 |
| beta-Defensin 3 | BMPR-II | BRCA1 |
| BRCA1 | N-Cadherin | PTEN |
| BTEB1 | CD44 | MSI1 |
| Calcitonin Gene-Related Peptide (CGRP) | CD45 | CD34 |
| Calcyclin | CD90/Thy1 | OCT-4 |
| Carcinoembryonic Antigen (CEA) | CD117/c-kit | SSEA |
| Cathepsin E (CaE) | CD45RO | CD133 |
| Caveolin-1 | Endoglin/CD105 | ABCG-2 |
| CD138 (Syndecan-1) | Fibronectin | SCA1 |
| CD151 | Fibronectin/Anastellin | STRO-1 |
| CD46 | HLA Class I | Nestin |
| Connexin-43 (Cx43) | ICAM-1/CD54 | PSA-NCAM |
| Muc-1 | Integrin alpha 1/CD49a | p75 Neurotropin |
| EMA | Integrin alpha 5/CD49e | N-Cadherin |
| Epithelial Sodium Channel-α | Integrin alpha V/CD51 | HLA Class1 |
| Epithelial Sodium Channel-β | Integrin beta 1/CD29 | CXCR4 |
| Epithelial Sodium Channel-γ | NCAM-1/CD56 | CSPG4 |
| Epithelial Sodium Channel-δ | NGF R/TNFRSF16 | CD38 |
| Epithelium specific antigen (EP-CAM, ESA) (AUA1) | Nucleostemin | CD90 |
| Epithelium/endothelial cells [PCX, Podocalyxin] | PDGF R alpha | CD117 |
| Exo-1 (Pa-G14) | Sca-1/Ly6 | CD146 |
| EZH2 | SSEA-4 | |
| Ezrin | STRO-1 | |
| Fas Ligand/TNFSF6 | TfR (Transferrin R) | |
| Fibrinogen (1F3) | VCAM-1/CD106 | |
| Foxa1 | Vimentin | |
| GABRP | Pi3KCA | |
| Galectin-3 | Snail | |
| Pan-Cytokeratin (AE1/AE3) | Twist | |
| Pan-Cytokeratin (KL1) | Slug | |
| GGT (gamma-glutamyl transpeptidase) | SMAD2/3 | |
| Glutamine Synthetase | AKt2 | |
| Heat Shock Protein 27 [HSP27] | cKit | |
| HLA-DR | cMet | |
| Lactoferrin | Notch | |
| LAMP-1 (lysosomal-associated membrane proprotein 1) | Sip1 | |
| Lectin | FOXC2 | |
| Lectins | SOX10 | |
| Leu-7 | beta Catenin | |
| MMR | VEGF | |
| MOC-31 | MMP2,3,9 | |
| NCAM-L1 (neural cell adhesion molecule L1) | mTOR | |
| Nectin-2/CD112 | cMet | |
| Normal Epithelial Cell Specific-1 (NES1)/kallikrein-10 | HER2/NEU | |
| NSE (neuron-specific enolase) | EGFR | |
| Ovarian Cancer Antigen [CA125] | MAGE3 | |
| p16 (INK4A) | | |
| P2X7 | | |
| p63 | | |
| P-Cadherin | | |
| plgR | | |
| Prominin-1 (CD133) | | |
| Prostasin/Prss8 | | |
| PSA | | |
| Prostatic Binding Protein (PBP) | | |
| Prosurfactant Protein B | | |
| Prosurfactant Protein C | | |
| PSCA (Prostate stem cell antigen) | | |
| Rab13 | | |
| RAGE | | |
| Rex-1 (zinc-finger protein-42, Zfp42) | | |
| Secretory Component (SC) | | |
| Sucrase-isomaltase (SI) | | |
| Surfactant Protein A | | |
| Surfactant Protein B | | |
| Surfactant protein C (SPC) | | |
| Surfactant Protein D | | |
| Survivin | | |
| TfR (Transferrin Rezeptor) | | |
| Transthyretin | | |
| UGRP1/SCGB3A2 | | |
| VAT-1 | | |
| PSMA | | |
| Cystatin C | | |
| Cytokeratin 10 | | |
| Cytokeratin 12 | | |
| Cytokeratin 13 | | |
| Cytokeratin 1-3 | | |
| Cytokeratin 14 | | |
| Cytokeratin 15 | | |
| Cytokeratin 16 | | |
| Cytokeratin 17 | | |
| Cytokeratin 18 | | |
| Cytokeratin 19 | | |
| Cytokeratin 20 | | |
| Cytokeratin 3 | | |
| Cytokeratin 4 | | |
| Cytokeratin 5 | | |
| Cytokeratin 5, 6 | | |
| Cytokeratin 7 | | |
| Cytokeratin 8 | | |
| Cytokeratin 8, 14, 18, 19 | | |
| Cytokeratin 8, 18 | | |
| Cytokeratin AE1 | | |
| Cytokeratin AE3 | | |
| Desmin | | |
| Desmocollin-2 | | |
| Desmocollin-3 | | |
| E-Cadherin | | |
| Ber-EP4 | | |
| Claudin-7 | | |

Diagnosing the resistance preferably comprises a step of comparing a measured expression level of ERCC1 isoform 3 protein and/or ERCC1 isoform 3 mRNA with a first predetermined value, wherein a measured expression level of ERCC1 isoform 3 protein and/or ERCC1 isoform 3 mRNA above the first predetermined value indicates a resistance against said therapeutic agent.

Diagnosing the resistance may comprise the steps of
a) comparing a measured expression level of ERCC1 isoform 3 protein and/or ERCC1 isoform 3 mRNA with a first predetermined value; and
b) comparing a measured expression level of at least one mRNA, which is different to ERCC1 isoform 3 mRNA, with a second predetermined value, wherein the at least one mRNA is selected from the group comprising or consisting of mRNAs which are markers for cells of epithelial, mesenchymal or stem cell phenotype, and
   wherein a measured expression level of ERCC1 isoform 3 protein and/or ERCC1 isoform 3 mRNA above the first predetermined value and a measured expression level of said at least one mRNA, which is different to ERCC1 isoform 3 mRNA, above the second predetermined value indicates a resistance against said therapeutic agent.

The at least one mRNA, which is different to ERCC1 isoform 3, may be selected from the group comprising or consisting of mRNAs recited in Table 1 (see above).

With reference to the following examples and figures, the subject-matter according to the invention is intended to be explained in more detail without wishing to restrict said subject-matter to the special embodiments shown here.

The FIGURE shows the results of the PCR assay validation by ROC curve analysis. Plotted is the sensitivity against the specificity of ERCC1 isoform 3 detection among all ERCC1 isoforms. More than 90% specificity (at 50% sensitivity) is obtained at a fragment concentration of approx. 0.2 ng/μl.

### EXAMPLE 1—PCR ASSAY VALIDATION BY ROC CURVE ANALYSIS

In the ROC curve analysis, the cut-off value corresponding to the specificity of the detection of ERCC1 isoform 3 by semi-quantitative PCR was determined.

cDNA was extracted from blood samples of 99 ovarian cancer patients and 21 healthy patients using the AdnaTest OvarianCancerSelect/Detect assay kit. Said cDNA potentially comprises all four isoforms of ERCC1. Subsequently, a fragment concentration analysis regarding the level of ERCC1 isoform 3 was performed using an Agilent 2100 Bioanalyzer (Agilent Technologies).

At a fragment concentration of approx. 0.2 ng/μl, a specificity for detection of ERCC1 isoform 3 among all ERCC1 isoforms of more than 90% was reached (see FIGURE).

In conclusion, PCR (e.g. semi-quantitative PCR) is a suitable method amongst others to specifically detect ERCC1 isoform 3 among all ERCC1 isoforms.

### EXAMPLE 2—PREDICTION OF PLATINUM RESISTANCE BY ERCC1 ISOFORM 3

In a clinical trial, blood samples of 143 pre-surgery ovarian cancer patients were analysed for the presence of circulating tumor cells (CTC) using the AdnaTest OvarianCancerSelect/Detect assay kit. Subsequently, all 143 patients received surgery followed by platinum-based chemotherapy.

Analysis of CTC was performed by immunomagnetic CTC enrichment targeting the epitopes of epithelial cell adhesion molecule (EPCAM) (also known as GA733-2) and mucin 1, cell surface associated (MUC1) and subsequently conducting a multiplex reverse-transcription PCR to detect the transcripts EPCAM, MUC1, and mucin 16, cell surface associated (MUC16) (also known as CA125), including, in a separate approach, ERCC1 isoform 3 transcripts.

The presence of CTC was observed in 14% of the patient samples. In said 14 CTC positive patient samples, ERCC1 isoform 3 expression was found to be positive in 57% of the analysed samples. Thus, in relation to all of the 143 analysed patient samples (i.e. in relation to all 143 patients), ERCC1 isoform 3-positive CTC (CTC ERCC1+) were observed in 8% of the samples (i.e. in 8% of all the 143 patients).

After the 143 patients received platinum-based chemotherapy, a multivariate log regression analysis was performed. ERCC1 isoform 3-positive CTC (CTC ERCC1+) were identified as independent predictive factor for platinum resistance as well as an independent prognostic factor for disease-free survival (DFS) (P=0.012 and P=0.007, respectively). The results of the analysis are shown in the Table 2 below.

TABLE 2
“ERCC1” refers to ERCC1 isoform 3, “DFS” refers to
disease-free survival, “OS” refers to overall survival, “HR”
refers to hazard ratio, “Figo” refers to the clinical Figo
staging, “pN” refers to lymph node stage, “M” refers
to metastatic stage, “G” refers to Grading, “RTB” refers
to “remaining tumor burden”, “CTC pre” refers to circulating
tumor cells before therapy, “CTC post” refers to circulating
tumor cells after therapy, “CTC (ERCC1+)” refers to CTC
which overexpress ERCC1 isoform 3 mRNA.

| | | | | | Platinum | |
|---|---|---|---|---|---|---|
| | | | | | resistance | |
| | | | | | (log. | |
| Cox | DFS | | OS | | Regression) | |
| Regres- | Indepen- | | Indepen- | | Indepen- | |
| sion | dent | HR | dent | HR | dent | HR |
| Figo | <0.0005 | 10.5 | 0.02 | 3.6 | <0.0005 | 10.5 |
| pN | * | * | * | * | * | * |
| M | <0.0005 | 3.9 | 0.026 | 2.3 | <0.0005 | 3.91 |
| G | <0.0005 | 0.29 | * | * | <0.0005 | 0.29 |
| RTB | * | * | <0.0005 | 2.0-7.9 | * | * |
| CTC pre | * | * | * | * | * | * |
| CTC post | * | * | * | * | * | * |
| CTC | 0.007 | 3.6 | * | * | 0.0012 | 3.58 |
| (ERCC1+) | | | | | | |

In conclusion, at primary diagnosis of ovarian cancer, the presence of ERCC1 isoform 3 in CTC can serve as a blood-based diagnostic biomarker for predicting platinum resistance as well as for providing a prognosis for a disease-free survival.

1-2. (canceled)

3. A method for diagnosing a resistance against a therapeutic agent in a patient, comprising:
a) measuring an amount of ERCC1 isoform 3 protein and/or an amount of ERCC1 isoform 3 mRNA, which present in a liquid sample or a tissue sample obtained from the patient, and
b) comparing the measured amount to a predetermined value, wherein an amount above the predetermined value indicates resistance against said therapeutic agent.

4. (canceled)

5. The method according to claim 3, wherein the liquid sample or the tissue sample is from a human patient.

6. The method according to claim 5, wherein the obtained sample is selected from the group consisting of a body fluid, peripheral blood, sputum, ascites, lymph, urine, bone marrow, and a biopsy material from a tumor.

7. The method according to claim 3, wherein the therapeutic agent is a cancer therapeutic agent.

8. The method according to claim 7, wherein the cancer therapeutic agent is for treating a cancer is selected from the group consisting of a solid cancer, breast cancer, lung cancer, prostate cancer, pancreatic cancer, bladder cancer, gastric cancer, colon cancer, testicular cancer and neck cancer.

9. The method according to claim 3, wherein the sampled obtained from the patient is a liquid sample, wherein the patient is a human, and wherein the method further comprises a step of isolating circulating tumor cells in the liquid sample, and a step of determining the level of expression of ERCC1 isoform 3 protein and/or ERCC1 isoform 3 mRNA in the isolated circulating tumor cells.

10. The method according to claim 9, wherein the step of isolating circulating tumor cells comprises the steps of
a) mixing the liquid sample with a predetermined combination of at least two antibodies and/or antibody derivatives having binding affinity to circulating tumor cells; and
b) isolating complexes between i) the antibodies and/or antibody derivatives and ii) the circulating tumor cells isolated from the liquid sample.

11. The method according to claim 10, wherein said antibodies and/or antibody derivatives are coupled to
a) at least one magnetic or paramagnetic particle; and/or
b) at least one fluorescent molecule.

12. The method according to claim 10, wherein at least one of the two antibodies and/or antibody derivatives is selected from the group consisting of GP1.4, MOC-31, Ber-EP-4, HMPV.2, HMEIV.2, 8B6, E29, 10E9 (anti-HER2), 2D3 (anti-EpCAM) anti-cMET, anti-EGFR, anti-cKIT, anti-IGFR and 131-11741.

13. The method according to claim 9, further comprising
a) lysing said isolated circulating tumor cells,
b) measuring the amount of ERCC1 isoform 3 protein and/or ERCC1 isoform 3 mRNA, present in the lysed isolated circulating tumor cells, and
c) measuring the amount of at least one mRNA, which is present in the lysed isolated circulating tumor cells and is different to ERCC1 isoform 3 mRNA, wherein the at least one mRNA is a marker for cells of epithelial, mesenchymal or stem cell phenotype.

14. (canceled)

15. The method according to claim 3, further comprising
comparing a measured expression level of at least one mRNA, which is different to ERCC1 isoform 3 mRNA, with a second predetermined value, wherein the at least one mRNA is selected from the group consisting of mRNAs which are markers for cells of epithelial, mesenchymal or stem cell phenotype, and
wherein a measured expression level of said ERCC1 isoform 3 protein and/or said ERCC1 isoform 3 mRNA above the first predetermined value and a measured expression level of said at least one mRNA, which is different to ERCC1 isoform 3 mRNA, above the second predetermined value indicates a resistance against said therapeutic agent.

16. The method according to claim 7, wherein the cancer therapeutic agent is a platinum-based cancer therapeutic agent, a radiation-based cancer therapeutic agent or a DNA-destructive cancer therapeutic agent.

17. The method according to claim 8, wherein the cancer therapeutic agent is for treating a solid cancer, and wherein the solid cancer is ovarian cancer.

18. The method according to claim 9, wherein the step of isolating circulating tumor cells in the liquid sample is performed by a multi-antibody capturing method.

19. The method according to claim 11, wherein said antibodies and/or antibody derivatives are coupled to at least one magnetic or paramagnetic particle and magnetic force is used for isolation.

20. The method according to claim 11, wherein said antibodies and/or antibody derivatives are coupled to at least one fluorescent molecule and fluorescence activated cell sorting is used for isolation.

21. The method according to claim 13, wherein step b) is performed before step c).

22. The method according to claim 13, wherein step c) is performed before step b).

23. The method according to claim 13, wherein measuring in step b) and/or step c) is done by RT-PCR, LCR, NASBA, a hybridization method and/or a method containing the steps of PCR, digesting of a PCR product with restriction enzymes to fragments and analysis of the fragments.
